(19) United States
(12) Patent Application Publication
(10) Pub. No.: US 2011/0152212 A1
(43) Pub. Date: Jun. 23, 2011

(54) Aqueous Formulations

(76) Inventors: Nicholas John Crowther, West Yorkshire (GB); Donald Eagland, West Yorkshire (GB)

(21) Appl. No.: 12/528,375

(22) PCT Filed: Feb. 22, 2008
(86) PCT No.: PCT/GB08/00601

§ 371 Date: Feb. 24, 2011

(30) Foreign Application Priority Data
Feb. 24, 2007 (GB) 0703627.0
Jun. 14, 2007 (GB) 0711440.8

(51) Int. Cl. A61K 31/704; A61K 31/58; A61K 31/65; A61K 31/05; A61K 31/4164; A61P 31/10; A61P 31/04; A61P 35/00; A61P 17/10

(52) U.S. Cl. 514/34; 514/174; 514/152; 514/153; 514/176; 514/731; 514/399

(57) ABSTRACT

A method of preparing an aqueous formulation of an active material and such a formulation per se are described, wherein the active material has a very low solubility in water. The method may involve contacting active material in solid form with an optionally cross-linked water soluble polymer. The water soluble polymer is preferably a polyvinyl polymer with polyvinylalcohol being preferred. The polymer is optionally cross-linked. The method and formulation may be of particular utility in delivery of anti-cancer drugs or drugs via a pulmonary route.

This invention relates to aqueous formulations and particularly, although not exclusively, relates to aqueous formulations of active materials, such as pharmaceutically active materials, having low aqueous solubility and/or low rates of dissolution in water.

It is, of course, desirable for pharmaceutically active materials to be administrable in aqueous formulations. However, many materials are known which have such low water solubility that, disadvantageously, they cannot be administered in aqueous formulations. For example, doxorubicin is a known drug used in the treatment of cancer. It has a relatively low solubility in water and a low rate of dissolution in water. In practice, it is formulated at a concentration of 0.1 or 0.2 wt % in dimethylsulphoxide and injected into patients. The use of dimethylsulphoxide is undesirable. Similarly, doxycycline is a known acne drug which may be administered topically or by injection. It has a relatively low solubility in water and cannot readily be administered in an aqueous formulation.

It is an object of the present invention to address the above described problems.

According to a first aspect of the invention, there is provided a method of preparing an aqueous formulation of an active material, the method comprising:

(i) selecting an active material in a solid form; and

(ii) contacting said active material with an optionally cross-linked water soluble polymer.

Said active material may be one which is sparingly soluble or insoluble in water at 25° C. Advantageously, the method may be used to prepare aqueous formulations of active materials at concentrations close to or in excess of the solubility of the active material and/or to prepare formulations which are more stable (e.g. over a temperature range such as down to 0° C.) than they would be in the absence of said optionally cross-linked water soluble polymer.

The ratio of the amount of active material (in g/L) in said formulation divided by the solubility (in g/L) in water of said active material at 25° C. may be at least 0.9, suitably at least 0.95, preferably at least 1, more preferably greater than 1, especially greater than 1.2.

Said method may be used to form aqueous formulations of active materials more rapidly compared to the formation of aqueous formulations in the absence of said optionally cross-linked water soluble polymer.

Said active material may have a solubility in water at 25° C. of less than 3000 μg/mL, less than 2500 μg/mL, less than 2000 μg/mL, less than 1500 μg/mL, less than 1000 μg/mL, less than 500 μg/mL, less than 300 μg/mL, less than 250 μg/mL, less than 200 μg/mL, less than 150 μg/mL, less than 100 μg/mL. The solubility as aforesaid may be greater than 0.1 μg/mL, greater than 0.5 μg/mL or greater than 1 μg/mL.

Said active material may have a log S measured in water at 25° C. of less than 1, less than 0.5, less than 0, less than −1, less than −1.5, less than −2, less than −2.5, less than −3 or even less than −3.5. The log S may be greater than −9, greater than −8 or greater than −7.

Said active material may have a log P of greater than −1, greater than −0.75, greater than −0.5, greater than −0.25. The log P may be less than 10, less than 8, less than 6, less than 4 or less than 2.

Said aqueous formulation prepared in the method preferably comprises particles comprising active material and said optionally cross-linked water soluble polymer. Said particles may comprise active material complexed with said optionally cross-linked water soluble polymer. Such particles may have maximum diameters of less than 500 nm, less than 250 nm, less than 200 nm. Such particle sizes may be measured using a laser light scattering technique.

Particles in the aqueous formulation may have a size which is less than the wavelength of light so the formulations appears substantially clear.

The method may be applied to a wide range of different types of active materials.

Said active material may include a cyclic moiety. Said active material may include more than one cyclic moiety.

Cyclic moieties may include heteroatoms; but preferably do not include heteroatoms.

Said active material may include at least one aromatic moiety. A said aromatic moiety may include heteroatoms but preferably does not include heteroatoms.

Said active material may include one or more carbonyl moieties. Preferably, it includes at least 2, preferably at least 3 carbonyl moieties. It may include less than 5 carbonyl moieties.

Said active material may include one or more —OH moieties. Suitably, it includes at least 2, preferably at least 3, more preferably at least 4 —OH moieties. It may include less than 6 —OH moieties.

Said active material may include at least 2, preferably at least 3, more preferably at least 4 C═C moieties. The material may include less than 10, preferably less than 8 C═C moieties.

Said active material may include at least one —NH₂ moiety.

Said active material may be of formula

X₁-L₃-X₂

where X₁ and X₂ independently represent cyclic moieties and L₃ represents a linking moiety which may be bonded to X₁ in one or more positions and may be bonded to X₂ in one or more positions.

X₁ may represent an optionally-substituted phenyl moiety.

X₂ may represent an optionally-substituted saturated or unsaturated, 6-membered ring. For example, it may represent a cyclohexyl, cyclohexenyl or phenyl moiety.

Said active material may comprise two, preferably three, more preferably four fused rings.

Said active material may include a moiety

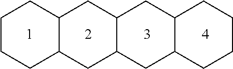

wherein each of rings 1 to 4 is optionally-unsaturated and optionally-substituted. Preferably, ring 1 is aromatic and at least one of rings 2 to 4 is unsaturated. Preferably, none of rings 1 to 4 is heteroaromatic.

Preferably, ring 1 includes a pendant —O— moiety, for example an —OH moiety.

Preferably, ring 2 includes at least one C═O moiety suitably wherein the carbon atom of the C═O moiety is an atom which is part of the ring structure of ring 2.

Preferably, ring 3 includes at least one, preferably at least two, —OH moieties, suitably at diametrically opposed positions on the ring.

Preferably, ring 4 includes a pendant —O— or —N< moiety.

Said active material may be an anthracycline or tetracycline.

Said active material may have a molecular weight of at least 100 g/mol, suitably at least 200 g/mol, preferably at least 300 g/mol, more preferably at least 400 g/mol. The molecular weight may be less than 800 g/mol, preferably less than 700 g/mol, more preferably less than 600 g/mol.

Said active material may be in a free form. Said active material is preferably not in a salt form.

Said active material may have a melting point of at least 50° C.

Said active material may be for treatment of cancer.

Said formulation may be for delivery using a nebuliser. Said formulation may be for pulmonary delivery.

Examples of water-soluble polymers include the following: water soluble gums, for example gum arabic, karaya gum, tragacanth gum, ghatti gum, guar gum; soybean derivatives, for example locust bean gum, tamarind gum; water soluble biopolymers, for example dextran, xanthan gum; water soluble proteins, for example gelatin type materials, carrageenan, agar and alginates, animal derivatives, casein, pectin; starch and starch derivatives, for example starch, modified starch, starch derivatives; cellulose derivatives, for example methyl cellulose, carboxymethyl cellulose, hydroxyethyl cellulose, hydroxypropyl cellulose; polyvinyls and maleic anhydride copolymers, for example polyvinyl alcohol, polyvinyl pyrrolidone; miscellaneous water soluble polyvinyls, for example maleic anhydride copolymers; polyacrylates and related systems, for example polyacrylates, polyacrylamides; polyimines and related systems, for example polyethylene oxides, polyethylenimines, polyethylene glycols; surface active water soluble polymers, for example lignosulfonates and related materials, lignites, tannins.

Preferably, said water-soluble polymer includes a functional group selected from an alcohol, carboxylic acid, carboxylic acid derivative, for example an ester, and an amine group. Said polymer preferably includes a backbone comprising, preferably consisting essentially of carbon atoms. The backbone is preferably saturated. Pendent from the backbone is suitably one or more said functional groups described. Said polymer may have a number average molecular weight (Mn) of at least 10,000, preferably at least 50,000, especially at least 75,000. Mn may be less than 500,000, preferably less than 400,000. Said polymer is preferably a polyvinyl polymer. Preferred polymers include optionally substituted, preferably unsubstituted, polyvinylalcohol, polyvinylacetate, polyalkylene glycols, for example polypropylene glycol, and collagen (and any component thereof) and of these polyvinylalcohol and/or polyvinylacetate based polymers are preferred. Examples of substituted polymers include polyvinylalcohol polymers which have been modified with hydrocarbon groups. Such polymers may be used in the method without them being cross-linked.

In one embodiment, preferred water-soluble polymers comprise a polymeric material which includes —O— moieties pendent from a polymeric backbone thereof. Said polymeric backbone of said polymeric material preferably includes carbon atoms. Said carbon atoms are preferably part of —CH₂— moieties. Preferably, a repeat unit of said polymeric backbone includes carbon to carbon bonds, preferably C—C single bonds. Preferably, said polymeric material includes a repeat unit which includes a —CH₂— moiety. Preferably, said polymeric backbone does not include any —O— moieties, for example —C—O— moieties such as are found in an alkyleneoxy polymer, such as polyethyleneglycol. Said polymeric backbone is preferably not defined by an aromatic moiety such as a phenyl moiety such as is found in polyethersulphones. Said polymeric backbone preferably does not include any —S— moieties. Said polymeric backbone preferably does not include any nitrogen atoms. Said polymeric backbone preferably consists essentially of carbon atoms, preferably in the form of C—C single bonds. Said —O— moieties are preferably directly bonded to the polymeric backbone. Said polymeric material preferably includes, on average, at least 10, more preferably at least 50, —O— moieties pendent from the polymeric backbone thereof. Said —O— moieties are preferably a part of a repeat unit of said polymeric material. Preferably, said —O— moieties are directly bonded to a carbon atom in said polymeric backbone of said polymeric material, suitably so that said polymeric material includes a moiety (which is preferably part of a repeat unit) of formula:

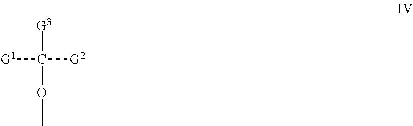

where G¹ and G² are other parts of the polymeric backbone and G³ is another moiety pendent from the polymeric backbone. Preferably, G³ represents a hydrogen atom. Preferably, said polymeric material includes a moiety

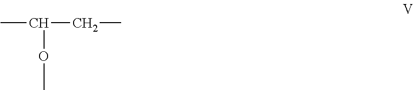

Said moiety V is preferably part of a repeat unit. Said moiety V may be part of a copolymer which includes a repeat unit which includes a moiety of a different type compared to moiety V. Suitably, at least 60 mole %, preferably at least 80 mole %, more preferably at least 90 mole % of said polymeric material comprises repeat units which comprise (preferably consist of) moieties V. Preferably, said polymeric material consists essentially of repeat units which comprise (preferably consist of) moieties V.

Suitably, 60 mole %, preferably 80 mole %, more preferably 90 mole %, especially substantially all of said polymeric material comprises vinyl moieties which are optionally cross-linked.

Preferably, the free bond to the oxygen atom in the —O— moiety pendent from the polymeric backbone of said polymeric material (and preferably also in moieties IV and V) is bonded to a group R¹⁰ (so that the moiety pendent from the polymeric backbone of said polymeric material is of formula —O—R¹⁰). Preferably group R¹⁰ comprises fewer than 10, more preferably fewer than 5, especially 3 or fewer carbon atoms. It preferably only includes atoms selected from carbon, hydrogen and oxygen atoms. R¹⁰ is preferably selected from a hydrogen atom and an alkylcarbonyl, especially a methylcarbonyl group. Preferably moiety —O—R¹⁰ in said polymeric material is an hydroxyl or acetate group.

Said polymeric material may include a plurality, preferably a multiplicity, of functional groups (which incorporate the —O— moieties described) selected from hydroxyl and acetate groups. Said polymeric material preferably includes a multiplicity of hydroxyl groups pendent from said polymeric backbone. Said polymeric material preferably includes a multiplicity of acetate groups pendent from the polymeric backbone.

Preferably, each free bond to the oxygen atoms in —O— moieties pendent from the polymeric backbone in said polymeric material, except for any free bonds which are involved in cross-linking the polymeric material, is of formula —O—R¹⁰ wherein each group —OR¹⁰ is selected from hydroxyl and acetate.

Preferably, said polymeric material includes a vinyl alcohol moiety, especially a vinyl alcohol repeat unit. Said polymeric material preferably includes a vinyl acetate moiety, especially a vinylacetate repeat unit. Polyvinylalcohol is generally made by hydrolysis of polyvinylacetate. Said polymeric material may comprise a 0-100% hydrolysed, preferably a 5 to 95% hydrolysed, more preferably a 60 to 90%, especially a 70 to 90% hydrolysed polyvinylacetate

Said polymeric material may have a number average molecular weight (Mn) of at least 10,000, preferably at least 50,000, especially at least 75,000. Mn may be less than 500,000, preferably less than 400,000. Said polymeric material is preferably a polyvinyl polymer. Said polymeric material may be a copolymer.

Said polymeric material is preferably a polyvinyl alcohol polymer or copolymer.

Said polymeric material may be a random or block copolymer.

Said polymeric material may include relatively hydrophilic regions and relatively hydrophobic regions. Certain polymeric materials may be more suitable for forming aqueous formulations of particular active materials than others. For example, relatively hydrophobic active materials may be contacted in the method with more hydrophobic polymeric materials. When the polymeric material is a polyvinylalcohol, such polymers having lower levels of hydroxy moieties and/or higher levels or acetate moieties may be relatively hydrophobic and therefore more suitable for use with more hydrophobic active materials.

Said active material may be contacted in the method with a polar material.

Said polar material may be reacted with said water-soluble polymer in the method, suitably to introduce polarity into the polymer and/or to increase polarity in regions of said polymer. Said polar material may be arranged to introduce a positive charge on said polymer.

Said polar material may include cationic moieties and suitably a counter ion.

Said polar material may include an N-containing moiety which may be an N⁺ moiety. The N-containing moiety may be part of an aromatic moiety. It may be part of an optionally-substituted pyridine-based moiety, for example an optionally-substituted N-coordination complex of pyridine. An N-coordination complex may include an optionally-substituted, preferably an unsubstituted, alkyl group bonded to the N-atom of the pyridine-based moiety.

Said polar material may include an aldehyde group, suitably as a substituent of an aromatic moiety, for example a phenyl moiety.

Said polar material may include a moiety of formula

X₃-L₂-X₄

wherein X₃ and X₄ are, independently, moieties which include polar atoms or groups and L₂ includes a linking atom or group.

Moiety L₂ may comprise an unsubstituted hydrocarbon moiety, for example a —CHR¹⁰—(CH₂)ₙ—CHR¹⁰— moiety or an unsaturated derivative thereof, where n is 0 or a positive integer for example, 0, 1, 2, 3 and R¹⁰ and R¹¹represent hydrogen atoms or linking atoms or groups. When R¹⁰ and R¹¹ include linking atoms or groups, such linking atoms or group both preferably include carbon atoms which are directly bonded to the cation atoms of respective —CHR¹⁰— and —CHR¹¹— moieties. Preferably L₂ comprises a >CH—CH< moiety wherein one free bond of each >CH moiety is bonded to X₃ and X₄.

X₃ may comprise said N-containing moiety described.

X₄ may comprise an aromatic moiety which is substituted by an aldehyde group as described.

Said polar material may itself be a polymeric material.

A cross-linking means as hereinafter described may define said polar material.

When said active material is contacted with a cross-linked water soluble polymer, said water soluble polymer may be cross-linked using a said cross-linking means.

A preferred cross-linking means comprises a chemical cross-linking material. Such a material is preferably a polyfunctional compound having at least two functional groups capable of reacting with functional groups of said water-soluble polymer. Preferably, said cross-linking material includes one or more of carbonyl, carboxyl, hydroxy, epoxy, halogen or amino functional groups which are capable of reacting with groups present along the polymer backbone or in the polymer structure of the hydrophilic polymer. Preferred cross-linking materials include at least two aldehyde groups. Thus, in a preferred embodiment, said active material is contacted with a material formed by cross-linking polyvinylalcohol using a material having at least two aldehyde groups.

Thus, said cross-linked water-soluble polymer may include a moiety of formula I:

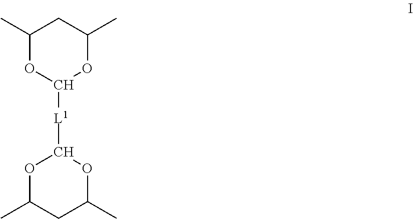

wherein L¹ is a residue of said cross-linking material.

Said cross-linking material preferably comprises a cross-linking polymeric material. Said cross-linking polymeric material preferably includes a repeat unit of formula

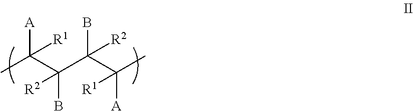

wherein A and B are the same or different, are selected from optionally-substituted aromatic and heteroaromatic groups and at least one comprises a relatively polar atom or group and R¹ and R² independently comprise relatively non-polar atoms or groups.

A and/or B could be multi-cyclic aromatic or heteroaromatic groups. Preferably, A and B are independently selected from optionally-substituted five or more preferably six-membered aromatic and heteroaromatic groups. Preferred heteroatoms of said heteroaromatic groups include nitrogen, oxygen and sulphur atoms of which oxygen and especially nitrogen, are preferred. Preferred heteroaromatic groups include only one heteroatom. Preferably, a or said heteroatom is positioned furthest away from the position of attachment of the heteroaromatic group to the polymer backbone. For example, where the heteroaromatic group comprises a six-membered ring, the heteroatom is preferably provided at the 4-position relative to the position of the bond of the ring with the polymeric backbone.

Preferably, A and B represent different groups. Preferably, one of A or B represents an optionally-substituted aromatic group and the other one represents an optionally-substituted heteroaromatic group. Preferably A represents an optionally-substituted aromatic group and B represents an optionally-substituted heteroaromatic group especially one including a nitrogen heteroatom such as a pyridinyl group.

Unless otherwise stated, optionally-substituted groups described herein, for example groups A and B, may be substituted by halogen atoms, and optionally substituted alkyl, acyl, acetal, hemiacetal, acetalalkyloxy, hemiacetalalkyloxy, nitro, cyano, alkoxy, hydroxy, amino, alkylamino, sulphinyl, alkylsulphinyl, sulphonyl, alkylsulphonyl, sulphonate, amido, alkylamido, alkylcarbonyl, alkoxycarbonyl, halocarbonyl and haloalkyl groups. Preferably, up to 3, more preferably up to 1 optional substituents may be provided on an optionally substituted group.

Unless otherwise stated, an alkyl group may have up to 10, preferably up to 6, more preferably up to 4 carbon atoms, with methyl and ethyl groups being especially preferred.

Preferably, A and B each represent polar atoms or group—that is, there is preferably some charge separation in groups A and B and/or groups A and B do not include carbon and hydrogen atoms only.

Preferably, at least one of A or B includes a functional group which can undergo a condensation reaction, for example on reaction with said water-soluble polymer. Preferably, A includes a said functional group which can undergo a condensation reaction.

Preferably, one of groups A and B includes an optional substituent which includes a carbonyl or acetal group with a formyl group being especially preferred. The other one of groups A and B may include an optional substituent which is an alkyl group, with an optionally substituted, preferably unsubstituted, C₁₋₄ alkyl group, for example a methyl group, being especially preferred.

Preferably, A represents a group, for example an aromatic group, especially a phenyl group, substituted (preferably at the 4-position relative to polymeric backbone when A represents an optionally-substituted phenyl group) by a formyl group or a group of general formula

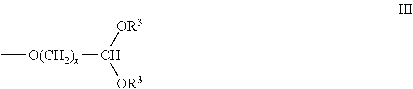

where x is an integer from 1 to 6 and each R³ is independently an alkyl or phenyl group or together form an alkalene group.

Preferably, B represents an optionally-substituted heteroaromatic group, especially a nitrogen-containing heteraromatic group, substituted on the heteroatom with a hydrogen atom or an alkyl or aralkyl group. More preferably, B represents a group of general formula

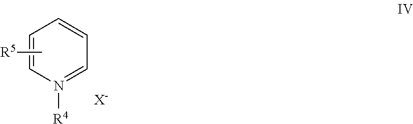

wherein R⁴ represents a hydrogen atom or an alkyl or aralkyl group, R⁵ represents a hydrogen atom or an alkyl group and X⁻ represents a strongly acidic ion. It may be an organic, for example alkyl, sulphate such a methylsulphate.

Preferably, R¹ and R² are independently selected from a hydrogen atom or an optionally-substituted, preferably unsubstituted, alkyl group. Preferably, R¹ and R² represent the same atom or group. Preferably, R¹ and R² represent a hydrogen atom.

Preferred cross-linking polymeric materials may be prepared from any of the following monomers by the method described in WO98/12239 and the content of the aforementioned document is incorporated herein by reference:

α-(p-formylstyryl)-pyridinium, γ-(p-formylstyryl)-pyridinium, α-(m-formylstyryl)-pyridinium, N-methyl-α-(p-formylstyryl)-pyridinium, N-methyl-β-(p-formylstyryl)-pyridinium, N-methyl-α-(m-formylstyryl)-pyridinium, N-methyl-α-(o-formylstyryl)-pyridinium, N-ethyl-α-(p-formylstyryl)-pyridinium, N-(2-hydroxyethyl)-α-(p-formylstyryl)-pyridinium, N-(2-hydroxyethyl)-γ-(p-formylstyryl)-pyridinium, N-allyl-α-(p-formylstyryl)-pyridinium, N-methyl-γ-(p-formylstyryl)-pyridinium, N-methyl-γ-(m-formylstyryl)-pyridinium, N-benzyl-α-(p-formylstyryl)-pyridinium, N-benzyl-γ-(p-formylstyryl)-pyridinium and N-carbamoylmethyl-γ-(p-formylstyryl)-pyridinium. These quaternary salts may be used in the form of hydrochlorides, hydrobromides, hydroiodides, perchlorates, tetrafluoroborates, methosulfates, phosphates, sulfates, methane-sulfonates and p-toluene-sulfonates.

Also, the monomer compounds may be styrylpyridinium salts possessing an acetal group, including the following:

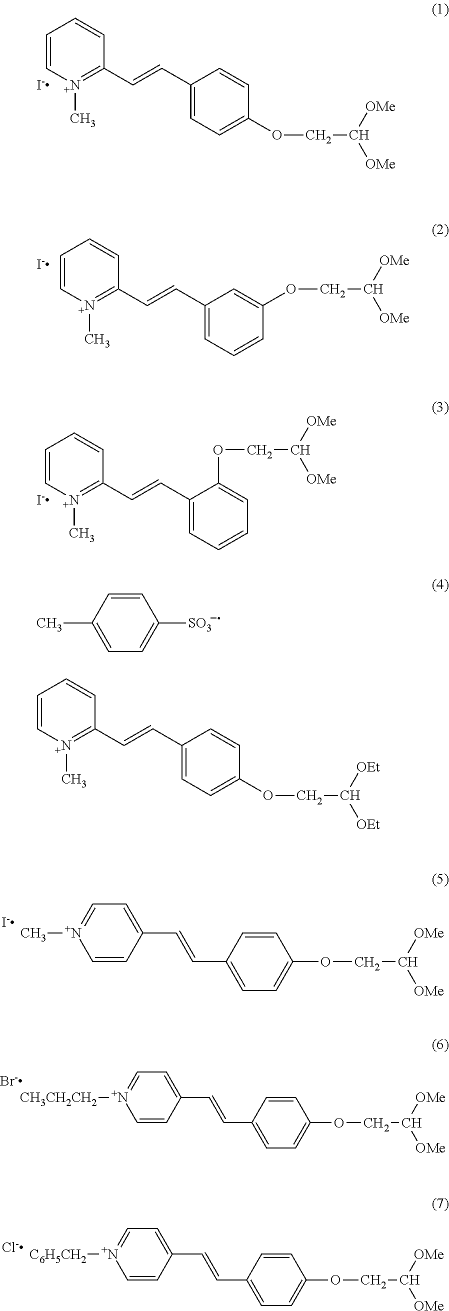

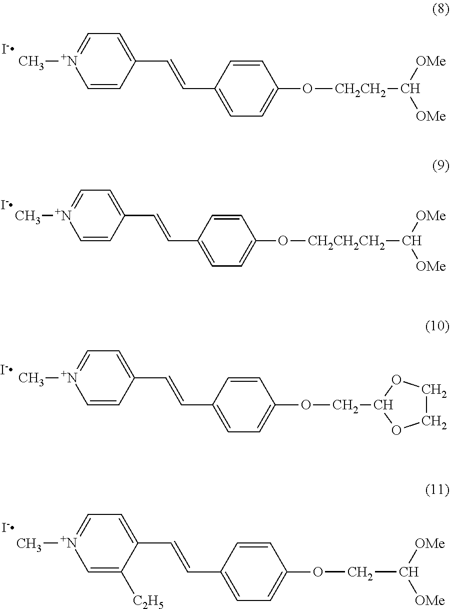

Thus, said cross-linking polymeric material is preferably prepared or preparable by providing a compound of general formula

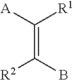

wherein A, B, R¹ and R² are as described above, in an aqueous solvent, (suitably so that molecules of said monomer aggregate) and causing the groups C═C in said compound to react with one another, (for example using UV radiation,) to form said cross-linking polymeric material.

Said cross-linking polymeric material may be of formula

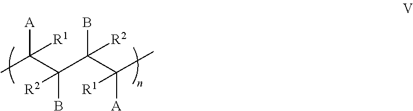

wherein A, B, R¹ and R² are as described above and n is an integer. Integer n is suitably 50 or less, preferably 20 or less, more preferably 10 or less, especially 5 or less. Integer n is suitably at least 1, preferably at least 2, more preferably at least 3.

When the method of the first aspect uses a cross-linked water-soluble polymer, the method may include a step of selecting a water soluble polymer as described above, selecting a cross-linking material as described above and contacting the two selected materials under conditions wherein the cross-linking material can cross-link the water-soluble polymer. The ratio of the wt % of selected water-soluble polymer to the wt % of selected cross-linking material may be 10 or less. The method may include reducing the pH of a formulation of said water-soluble polymer and said cross-linking material. Subsequently, the formulation may be neutralised.

The aqueous formulation prepared according to said first aspect may include less than 1 wt % or less than 0.5 wt % of said active material. It suitably includes at least 0.05 wt %, preferably at least 0.1 wt %, more preferably at least 0.15 wt % of said active material. Said formulation may include more than 60 wt %, suitably more than 75 wt %, preferably more than 95 wt %, more preferably more than 96 wt %, especially more than 97 wt % of water. The amount of water may be less than 99 wt %, preferably less than 98 wt %.

Said aqueous formulation suitably includes less than 5 wt % of organic polymeric materials (for example said water-soluble polymer and/or said cross-linking polymeric material and/or a reaction product thereof), preferably less than 4 wt %, more preferably less than 3 wt %. The aqueous formulation may include at least 1 wt %, preferably at least 1.5 wt %, more preferably at least 2 wt %, of organic polymeric materials. At least some, suitably at least 50 wt %, preferably at least 75 wt %, more preferably at least 90 wt %, of said organic polymeric material is selected from the group comprising polyvinylalcohol and cross-linked polyvinylalcohol. In said aqueous formulation, the ratio of the sum of the wt % of organic polymeric materials to the wt % of said active material is suitably at least 3, is preferably at least 6 and, more preferably, is at least 9. The ratio may be less than 50, preferably less than 30.

Said aqueous formulation may comprise a solvent phase which consists essentially of water. However, in some embodiments, the solvent phase may be modified by a modifying means. Said modifying means may be selected to increase the solubility of a selected active material in the solvent phase. Said modifying means may be a solvent. Said modifying means may include one or more hydroxy groups. Said modifying means is preferably pharmacologically acceptable. Said modifying means preferably comprises ethanol.

The ratio of the wt % of water to the wt % of modifying means (especially ethanol) in the solvent phase is suitably at least 0.5, preferably at least 0.75, especially at least 0.9.

When the solvent phase includes a modifying means (especially ethanol), the ratio of weights of water:modifying means may be at least 0.4, preferably at least 0.75, more preferably at least 0.9. The ratio is suitably less than 2, preferably less than 1.5, more preferably less than 1.2. Preferably, the ratio is in the range 0.8 to 1.2.

Suitably, said aqueous formulation comprises:
0.05 wt % to 1 wt % of a said active material;
1 wt % to 5 wt % of organic polymeric materials; and
94 wt % to 98.95 wt % of a solvent formulation which includes at least 45 wt % water and up to 55 wt % ethanol.

Suitably said aqueous formulation comprises:
0.05 wt % to 1 wt % of a said active material;
1 wt % to 5 wt % of organic polymeric materials; and
94 wt % to 98.95 wt % of water.

In the method, preferably a said water soluble polymer is selected and contacted with a said cross-linking material, suitably with mixing, in the presence of water, suitably in excess of 95 wt % water. Contact and/or mixing may be carried out at a temperature in the range 15 to 40° C., suitably at ambient temperature. The pH of the mixture may be arranged to be in the range 2-4. Subsequently, a said active material, suitably in a solid form, may be added to the mixture, suitably with stirring. The active material may be added when the pH is less than 7, less than 5 or in the range 2-4.

Said aqueous formulation is suitably a liquid.

According to a second aspect of the invention, there is provided an aqueous formulation prepared in a method according to the first aspect.

According to a third aspect of the invention, there is provided an aqueous formulation comprising an active material and an optionally cross-linked water soluble polymer.

The aqueous formulation of the second or third aspects may have any feature of the aqueous formulation of the first or second aspects.

In a preferred embodiment, the aqueous formulation includes 0.05 to 1 wt % of active material, 1 to 5 wt % of optionally cross-linked water soluble polymer (e.g. optionally cross-linked polyvinylalcohol especially a 70 to 90% hydrolysed polyvinylacetate) and 94 to 98.95 wt % of a solvent formulation which includes at least 45 wt % water and up to 55 wt % ethanol.

In a preferred embodiment, said water-soluble polymer is cross-linked, suitably by a cross-linking material which includes a repeat unit of formula II.

According to a fourth aspect of the invention, there is provided a pharmaceutical delivery means, for example composition, which includes an aqueous formulation according to the second or third aspects.

According to a fifth aspect of the invention, there is provided a method of treatment of the human or animal body which comprises administering to the body an aqueous formulation according to the second, third or fourth aspects.

According to a sixth aspect of the invention, there is provided the use of an aqueous formulation according to the second, third or fourth aspects as a medicament.

Said formulation is preferably for pulmonary delivery and/or is for treating a pulmonary condition. Said formulation is preferably for use in a nebuliser. Said active agent is preferably an anti-asthma drug.

According to a seventh aspect of the invention, there is provided the use of an aqueous formulation according to the second, third or fourth aspects in the manufacture of a medicament for treatment of a human or animal condition, for example disease. The condition may be cancer or acne.

According to an eighth aspect of the invention, there is provided an aqueous formulation comprising an active material dispersed in a solvent formulation which comprises water and ethanol, wherein the ratio of the weights of water: ethanol is at least 0.4 and less than 2, and, more preferably, is in the range 0.8 to 1.2.

Said active material may be as described in any statement herein.

Any feature of any aspect of any invention or embodiment described herein may be combined with any feature of any aspect of any other invention or embodiment described herein mutatis mutandis.

Specific embodiments of the invention will now be described, by way of example.

The following materials are referred to hereinafter:

Poval 220—a polyvinylalcohol obtained from Kuraray having a viscosity, measured on a 4% aqueous solution at 20° C. (determined by a Brookfield synchronised-meter rotary-type viscometer), of 30.mPa·s and a degree of hydrolysis (saponification) of about 88% mol %. The molecular weight is about 130,000.

KH-20 refers to a polyvinyl alcohol, obtained from Marubeni Speciality Chemicals, Inc, having a viscosity of 44-52 mPa·s and a degree of hydrolysis of 78.5-81.5 mol.

### EXAMPLE 1

### Preparation of poly(1,4-di(4-(N-methylpyridinyl))-2,3-di(4-(1-formylphenyl)butylidene

This was prepared as described in Example 1 of PCT/GB97/02529, the contents of which are incorporated herein by reference. In the method, an aqueous solution of greater than 1 wt % of 4-(4-formylphenylethenyl)-1-methylpyridinium methosulphonate (SbQ) is prepared by mixing the SbQ with water at ambient temperature. Under such conditions, the SbQ molecules form aggregates. The solution was then exposed to ultraviolet light. This results in a photochemical reaction between the carbon-carbon double bonds of adjacent 4-(4-formylphenylethenyl)-1-methylpyridinium methosulphate molecules (I) in the aggregate, producing a polymer, poly (1,4-di(4-(N-methylpyridinyl))-2,3-di(4-(1-formylphenyl)butylidene methosulphonate (II), as shown in the reaction scheme below. It should be appreciated that the anions of compounds I and II have been omitted in the interests of clarity.

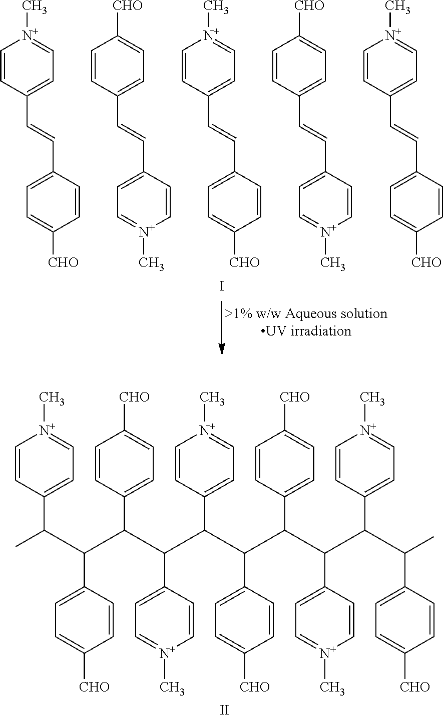

### EXAMPLE 2

### General Procedure for Preparation of Formulation of Drug of Very Low Water Solubility

An aqueous mixture is prepared comprising water containing up to 2.5 wt % of polyvinylalcohol and the butylidene polymer of Example 1 at up to 10 wt % of the amount of polyvinylalcohol in the mixture. The mixture may be prepared by mixing the materials described at ambient temperature. The aqueous mixture is acidified to a pH value of not less than 2.5 which causes the polyvinylalcohol and butylidene polymer to react in an acid catalysed reaction. Subsequent to the reaction the pH of the mixture may be raised to about 7.4 by addition of alkali.

A drug of low water solubility, in a solid form (e.g. a powderous form), may be added to the mixture of polyvinylalcohol and butylidene polymer after the pH has been lowered to not less than 2.5. In one embodiment, it may be added whilst the pH of the mixture is at or about its lowest level; in other embodiments it may be added after the pH has been raised to about 7.4.

In the method, the polyvinylalcohol and butylidene polymer react in an acid catalysed reaction to produce a hydrogel layer which it is believed is able to stabilise solid drug molecules which therefore appear to be dissolved in the water (although strictly speaking the drug molecules may not form a true solution). Particle size assessment of formulations prepared suggests that particles comprising the drug molecules stabilised by the polyvinylalcohol/butylidene polymer reaction product are formed. Formation of the hydrogel is summarised in the scheme below.

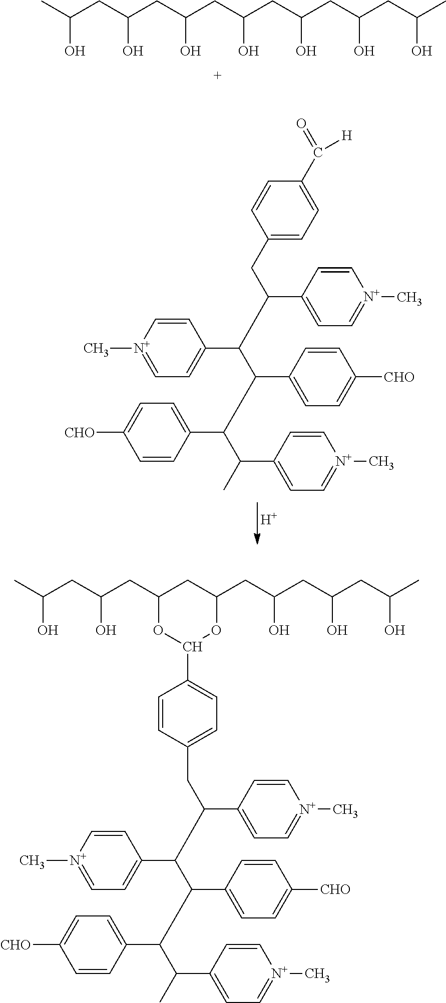

### EXAMPLE 3

### Preparation of Aqueous Formulation of Doxorubicin

A 0.5 wt % formulation of a hydrogel was prepared comprising Poval 220 polyvinylalcohol and the butylidene polymer of Example 1 in the weight ratio 10:1. The pH of the mixture was lowered to about 2.5 by addition of hydrochloric acid and, with the pH at the lowered level, doxorubicin, a solid red powder, was added at ambient temperature with stirring to give a level of 0.2 wt % doxorubicin in the mixture. The doxorubicin appeared to dissolve in the water substantially instantaneously to give a clear red mixture which appears to comprise a solution but more likely comprises doxorubicin which is complexed by the particles of hydrogel formed from the polyvinylalcohol and butylidene polymer. After the formulation has been prepared, the pH may be raised to about 7 by addition of alkali to neutralise it.

It is found that an aqueous formulation having a saturation solubility of 115 mg/ml (11.5% w/v) can be prepared using the aforementioned process.

The formulation may be administered orally/intravenously or in a pulmonary manner and is found to maintain its medical efficacy.

### EXAMPLE 4

### Preparation of Aqueous Formulation of Doxycycline

A 2 wt % formulation of a hydrogel was prepared comprising KH-20 polyvinylalcohol and the butylidene polymer of Example 1 in a weight ratio 10:1. The pH of the mixture was lowered to about 2.5, by addition of hydrochloric acid. Then, doxycycline, in a solid form, was added at ambient temperature with stirring to give a level of 0.5 wt % of doxycycline in the mixture. The doxycycline appeared to dissolve in the water within about 15 minutes to give a clear pale yellow mixture which appeared to comprise a solution but more likely comprises complexed doxycycline.

The formulation may be administered orally/intravenously or topically and is found to maintain its medical efficacy.

### EXAMPLE 5

### Preparation of Aqueous Formulation of Budesonide

The anti-asthma drug budesonide has a very low solubility. By a process analogous to those described above, a solution of the drug at a concentration in excess of 250 μg/ml can be prepared which significantly exceeds the concentration attainable by simple dissolution in water.

The procedures described may be used to disperse a range of drugs or other active ingredients in solid form suitably to produce formulations comprising 0.1 to 1.5 wt % of active ingredient. In some cases, selecting different types of polyvinylalcohol may affect the ability to disperse the active ingredients. For example, when an active ingredient is particularly hydrophobic, a polyvinyl alcohol having a greater concentration of hydrophobic acetate moieties (i.e. a polyvinylalcohol having a lower hydrolysis level) may be used.

The procedure may be used for active ingredients which are sparingly soluble in water and/or wherein the solubility in water is low or the rate of dissolution is disadvantageously slow.

The procedure may be used to prepare formulations of drugs for use in nebulisers. This is illustrated by Example 6.

### EXAMPLE 6

### Aerosolisation of Concentrated Budesonide Solution

Solutions containing budesonide for aeroslisation are generally limited to suspensions because of its physiochemical properties. Using the process described in Example 5 an 8 mg ml⁻¹ solution of budesonide was prepared with a mean particle size of 400.2 nms (measured using a “Zetapals” instrument, Brookhaven Instruments Corporation) Nebulisation of this solution was compared to that of a commercially available budesonide suspension (Pulmicort Respules, AstraZeneca) containing 250 μg ml⁻¹ of budesonide using CEN methodology (EN-13544-1). 4 mls of each solution was nebulised using a PARI-LC-Plus nebuliser set attached to a Pariboy compressor (Pari, GmbH). Budesonide output rather than that of a sodium fluoride tracer was measured. Results are provided in table 1 below.

TABLE 1
Mean (SD) aerodynamic characteristics of the emitted dose (n = 3)

| | |
|---|---|
| Solution prepared | Pulmicort Respules |
| as in Example 5 | Suspension |

| | | | |
|---|---|---|---|
| Total aerosol output | 23.1 | (0.3) | 0.88 (0.06) |
| (mg) | | | |
| T-piece and | 3.4 | (0.2) | 0.05 (0.01) |
| attachments (mg) | | | |
| Chamger (mg) | 6.1 | (0.1) | 0.07 (0.01) |
| Nebulisation time | 5.14 | (0.03) | 5.44 (0.02) |
| (min) | | | |
| Fine particle fraction | 47.4 | (0.5) | 48.9 (0.8) |
| (%) | | | |
| MMAD (μm) | 3.72 | (0.04) | 3.49 (0.04) |
| GSD (no units) | 2.76 | (0.04) | 2.81 (0.03) |

In the first 3 minutes the % total emitted dose for the solution prepared as in Example 5 and Pulmicort Respules Suspension was 68.8 and 38.6% (nominal dose), respectively. The results highlight the potential of formulations described herein to aerosolise concentrated solutions of poorly soluble drugs and to deliver smaller volumes of therapeutic doses in suitable inhalers.

### EXAMPLE 7

### Alternative Formulations for Drugs of Low Water Solubility

As an alternative to the general procedure described in Example 2 and the procedures described in other examples, a solvent may comprise a 50:50 wt % water/ethanol solution. The polyvinylalcohol and butylidene polymer may be included in such a solvent and the mixture may then be acidified to a pH of not less than 2.5 to cause polyvinylalcohol and butylidene polymer to react in an acid catalysed reaction. A drug of low solubility may then be added to the mixture as described in the other examples.

Table 2 below details solubilities for selected drugs in the formulation described (“Example 7 formulation” in the table) and compares solubility to the intrinsic solubility of the drugs and the solubility in a 10% w/v hydroxypropyl cyclodextrin solution, following the procedure described in T. Loftsson et al, Int. J. Pharm 302(2005), 18-28 and S. I. F. Badaway et al, Int. J. Pharmaceut.128(1996), 45-54. The table illustrates that relatively concentrated solutions can be obtained using Example 7 formulations.

| | | Solubility in | |
|---|---|---|---|
| | | 10% w/v | |
| | Intrinsic | Hydroxypropyl | Example 7 |
| | Solubility | Cyclodextrin | formulation |
| Drug | (mg/ml) | soln. (mg/ml) | (mg/ml) |
| Clotrimazole | 0.07 | 1.21 | 11.8 |
| Econazole | 0.37 | 4.99 | 12.6 |
| Miconazole | 0.09 | 2.46 | 4.7 |
| Propafol | 0.16 | 7.69 | 34.8 |
| Danazol | 0.001 | 0.034 | 1.9 |

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A method of preparing an aqueous formulation of an active material, the method comprising:
selecting an active material in a solid form; and
(ii) contacting said active material with an optionally cross-linked water soluble polymer.

2. A method according to claim 1, wherein the ratio of the amount of active material, in g/L, in said formulation divided by the solubility, in g/L, in water of said active material at 25° C. is greater than 1.2.

3. A method according to claim 1, wherein said active material has a solubility in water at 25° C. of less than 3000 μg/mL.

4. A method according to claim 1, wherein said active material has a log S measured in water at 25° C. of less than 0; and a log P of greater than −0.25.

5. A method according to claim 1, wherein said active material includes a cyclic moiety, and has a molecular weight of at least 200 g/mol.

6. (canceled)

7. A method according to claim 1, wherein said active material is not in a salt form.

8. A method according to claim 1, wherein said active material is for treatment of cancer; or said formulation is for pulmonary delivery.

9. A method according to claim 1, wherein said water-soluble polymer includes a functional group selected from an alcohol, carboxylic acid, carboxylic acid derivative and an amine group.

10. A method according to claim 1, wherein said water-soluble polymer comprises a polymeric material which includes —O— moieties pendent from a polymeric backbone thereof.

11. A method according to claim 10, wherein said polymeric backbone consists essentially of carbon atoms; and said —O— moieties are directly bonded to the polymeric backbone.

12. A method according to claim 1, wherein said polymeric material includes a repeat unit comprising a moiety

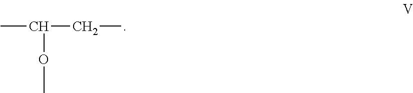

13. (canceled)

14. A method according to claim 1, wherein said polymeric material comprises a 0-100% hydrolysed polyvinyl acetate.

15. A method according to claim 1, wherein said polymeric material is a polyvinyl alcohol polymer or copolymer.

16. (canceled)

17. A method according to claim 1, wherein said water soluble polymer is cross-linked and includes a moiety of formula 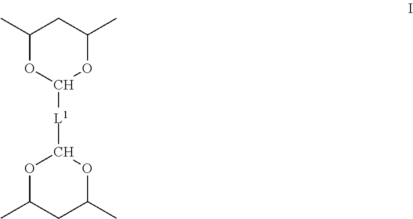
wherein L¹ is a residue of a cross-linking material.

18. A method according to claim 17, wherein said cross-linking material includes a repeat unit of formula 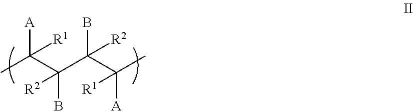
wherein A and B are the same or different, are selected from optionally-substituted aromatic and heteroaromatic groups and at least one comprises a relatively polar atom or group and R¹ and R² independently comprise relatively non-polar atoms or groups.

19. A method according to claim 1, wherein said aqueous formulation comprises a solvent phase which consists essentially of water; or said aqueous formulation comprises a solvent phase which is modified by a modifying means which includes one or more hydroxy groups, wherein the ratio of the wt % of water to the wt % of modifying means in the solvent phase is at least 0.5.

20. (canceled)

21. A pharmaceutical delivery means comprising an aqueous formulation comprising an active material and an optionally cross-linked water-soluble polymer.

22. A pharmaceutical delivery means according to claim 21, wherein the aqueous formulation comprises
0.05 wt % to 1 wt % of a said active material;
1 wt % to 5 wt % of organic polymeric materials; and
94 wt % to 98.95 wt % of a solvent formulation which includes at least 45 wt % water and up to 55 wt % ethanol.

23. (canceled)

24. A method of treatment of the human or animal body which comprises administering to the body a pharmaceutical delivery means according to claim 21.

25. (canceled)

26. A method according to claim 24, said method comprising pulmonary delivery; treatment of a pulmonary condition, cancer or acne.

27. (canceled)
